(19) United States
(12) Patent Application Publication
(10) Pub. No.: US 2012/0046551 A1
(43) Pub. Date: Feb. 23, 2012

(54) ULTRASONIC PROBE

(75) Inventor: Shengli Tang, Shenzhen (CN)

(73) Assignee: SHENZHEN MINDRAY BIO-MEDICAL ELECTRONICS CO., LTD., Shenzhen (CN)

(21) Appl. No.: 13/195,631

(22) Filed: Aug. 1, 2011

(30) Foreign Application Priority Data
Aug. 18, 2010 (CN) 201010256583.6

(51) Int. Cl. A61B 8/00

(52) U.S. Cl. 600/459

(57) ABSTRACT

A three-dimensional (3D) mechanical probe capable of ensuring reliable sealing between an acoustic window and a base is disclosed.

### CROSS-REFERENCE TO RELATED APPLICATIONS

This application claims the benefit of priority from Chinese Patent Application No. 201010256583.6, filed on Aug. 18, 2010, which is incorporated herein by reference in its entirety.

### TECHNICAL FIELD

The present disclosure relates generally to ultrasonic imaging and, more particularly, to a three-dimensional (3D) mechanical scanning probe for medical ultrasonic imaging.

### SUMMARY OF THE INVENTION

A three-dimensional (3D) mechanical probe may include an acoustic window, a base, a restricting body, and an elastic seal ring. The acoustic window and the base may be fixedly connected to form an enclosed space. The acoustic window may have a first matching wall, the base may have a second matching wall, and the first matching wall may be sleeved on the second matching wall. The seal ring may be disposed between the first matching wall and the second matching wall and may be tightly compressed and deformed, and the restricting body may restrict deformation of the acoustic window.

### BRIEF DESCRIPTION OF THE DRAWINGS

FIG. 1 is a schematic sectional view showing a connection relation between an acoustic window and a base in the prior art;

FIG. 2 is an enlarged view of part A in FIG. 1;

FIG. 3 is a schematic structural view of a 3D mechanical probe according to an embodiment (acoustic window not shown);

FIG. 4 is a schematic structural view of a 3D mechanical probe according to an embodiment;

FIG. 5 is a schematic sectional view of a 3D mechanical probe according to an embodiment;

FIG. 6 is an enlarged view of part B in FIG. 5;

FIG. 7 is a schematic structural view of an acoustic window and a restricting collar before being assembled according to an embodiment;

FIG. 8 is a schematic structural view of a restricting collar in a butted form;

FIG. 9 is a schematic structural view of a restricting collar in a joined form;

FIG. 10 is a schematic structural view of an acoustic window according to an embodiment;

FIG. 11 is a schematic structural view of a base according to an embodiment;

FIG. 12 is a schematic sectional view of an acoustic window, a base, and a restricting collar after being assembled together according to an embodiment; and

FIG. 13 is an enlarged view of part D in FIG. 12.

### DETAILED DESCRIPTION

An ultrasonic probe having a 3D imaging function is called a 3D mechanical probe, and generally has a stepping motor serving as a driving power source therein. The stepping motor is controlled by a signal to drive an acoustic head to swing within certain angles through a transmission system. At each swing angle, the 3D mechanical probe can function as a conventional probe to emit an ultrasonic wave and receive an echo carrying human tissue information, and thus can image human tissues at any angle within the swing range, without requiring a doctor to slide or swing the probe on the human body.

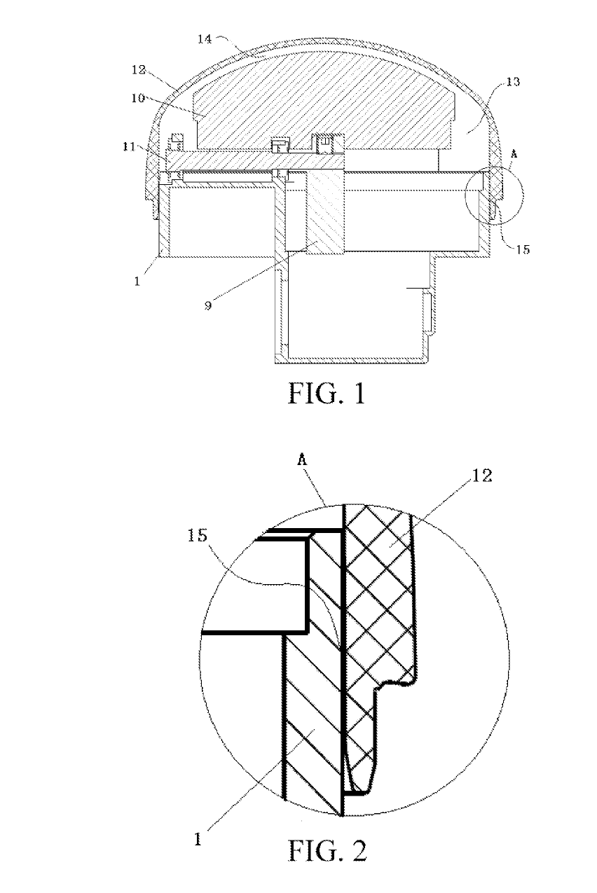

As shown in FIGS. 1 and 2, an existing 3D mechanical probe includes a base 1 and an acoustic window 12. The base 1 and the acoustic window 12 are connected to form an enclosed space 13. An acoustic head 10 is fixedly connected to a driven wheel 9 of a transmission system. The driven wheel 9 is supported on the base 1 through an acoustic-head rotating shaft 11 and is capable of free rotation. A stepping motor drives the transmission mechanism, such that the driven wheel 9 drives the acoustic head 10 to swing within the enclosed space 13. The enclosed space 13 is filled with a coupling liquid, and the coupling liquid is used for filling a gap 14 between the acoustic head and the acoustic window to conduct an ultrasonic wave. In order to prevent the coupling liquid from leaking from a junction surface 15 between the acoustic window and the base, the junction surface 15 requires reliable connection and sealing.

The connection and the sealing between the acoustic window 12 and the base 1 are generally realized by an adhesive. The acoustic window 12 is commonly made of a material, such as poly(4-methylpentene-1) (TPX), polypropylene (PP), or low-density polyethylene (LDPE). Such a material has a low surface energy and is difficult to be reliably adhered to. The adhesive is in contact with the coupling liquid for a long time, and when the acoustic head 10 swings, the coupling liquid in the enclosed space 13 exerts an oscillating pressure on the adhesion surface. Such factors easily cause stripping of an inner wall of the acoustic window 12 and the adhesive from the adhesion surface, and, as a result, the reliable connection and the reliable sealing between the acoustic window 12 and the base 1 cannot be ensured.

The present disclosure is directed to a 3D mechanical probe capable of ensuring reliable sealing between an acoustic window and a base.

To achieve the above objectives, one embodiment of a 3D mechanical probe includes an acoustic window, a base, a restricting body, and an elastic seal ring. The acoustic window and the base are fixedly connected to form an enclosed space. The acoustic window has a first matching wall, the base has a second matching wall, and the first matching wall is sleeved on the second matching wall. The seal ring is disposed between the first matching wall and the second matching wall and is tightly compressed and deformed, and the restricting body restricts deformation of the acoustic window.

By disposing the seal ring having an amount of compressive deformation between the first matching wall and the second matching wall, the sealing between the acoustic window and the base can be effectively ensured, thereby preventing leakage of a coupling liquid; by disposing the restricting body, the acoustic window can be prevented from being expanded by the seal ring, thereby ensuring a sufficient amount of deformation of the seal ring, and further ensuring the sealing between the base and the acoustic window. The base may be in a snap-fit engagement with the acoustic window, thereby ensuring a reliable connection therebetween.

The present disclosure is further described in detail through embodiments with reference to the accompanying drawings.

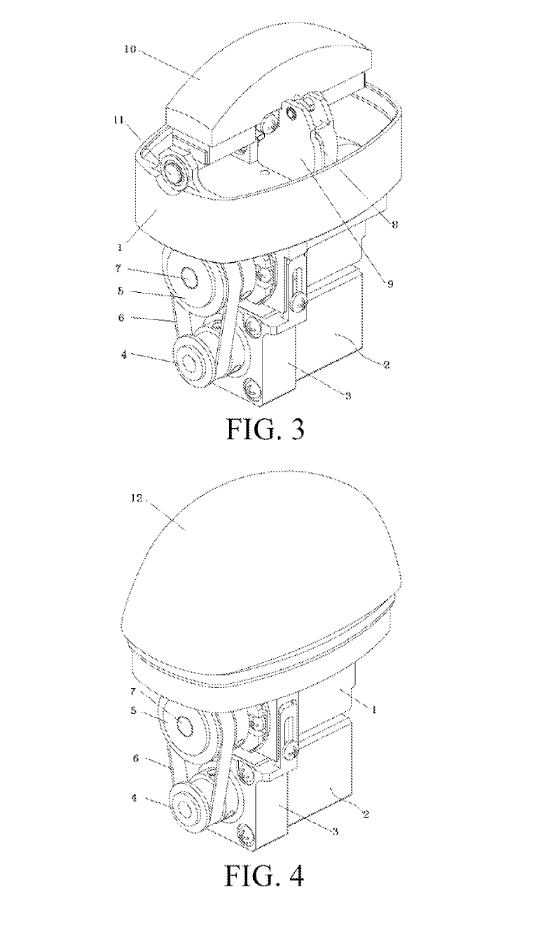

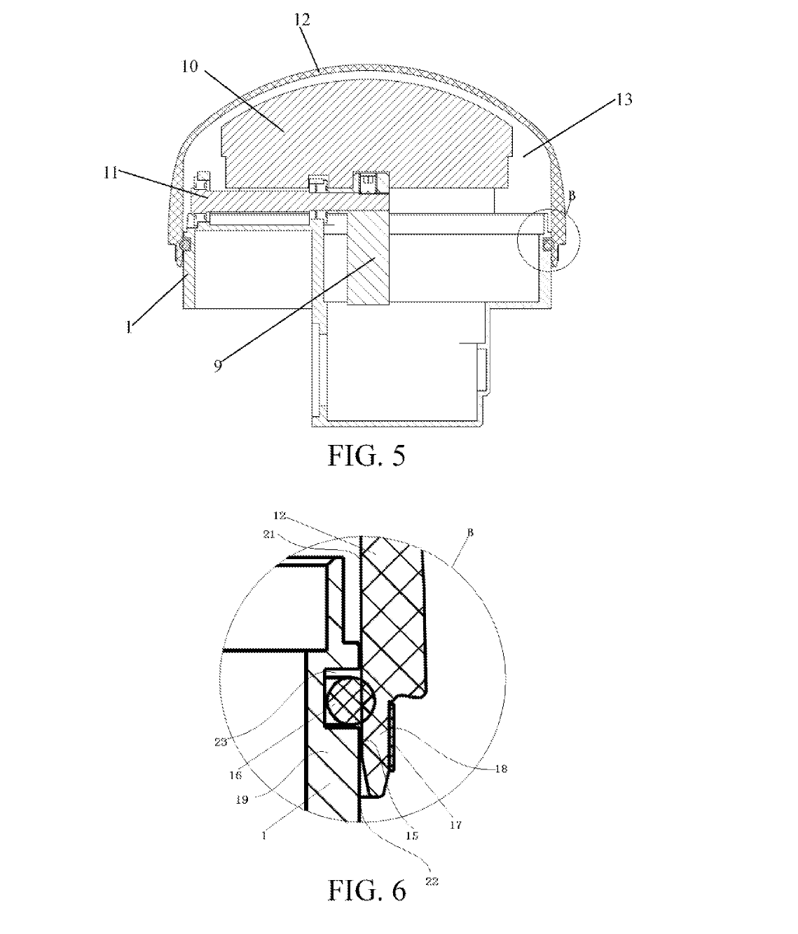

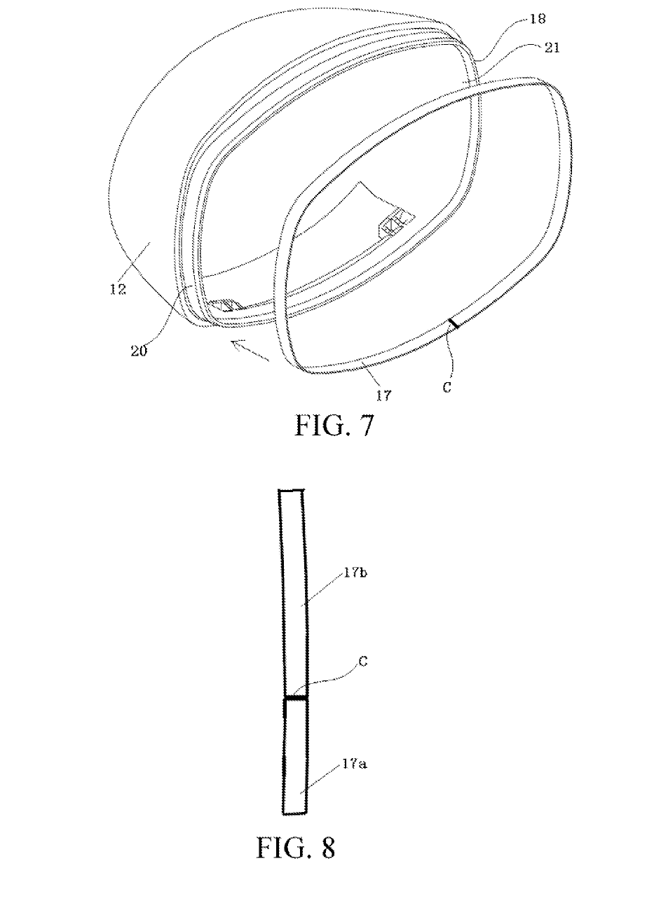

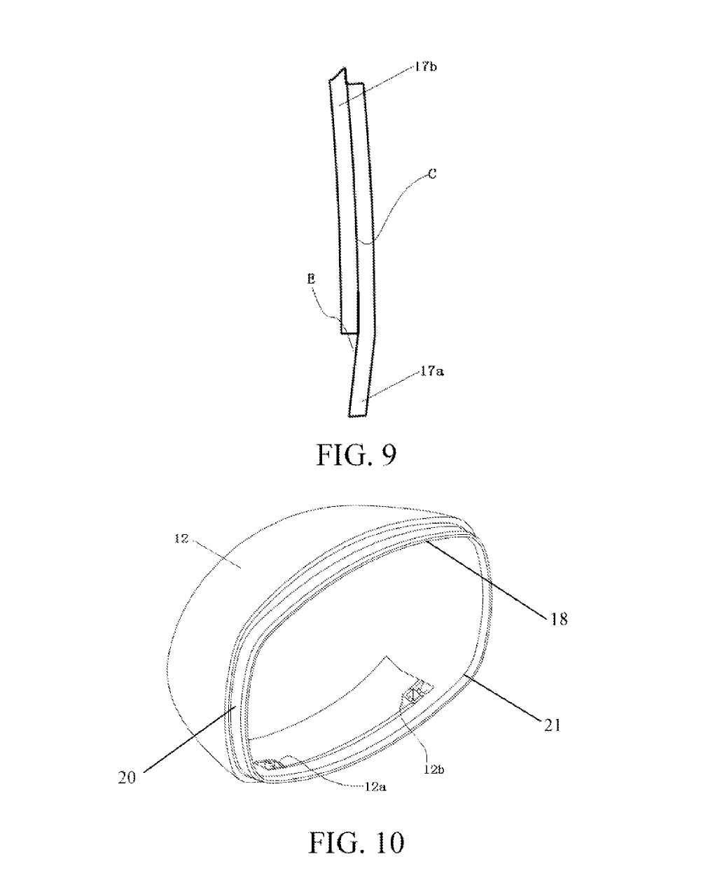

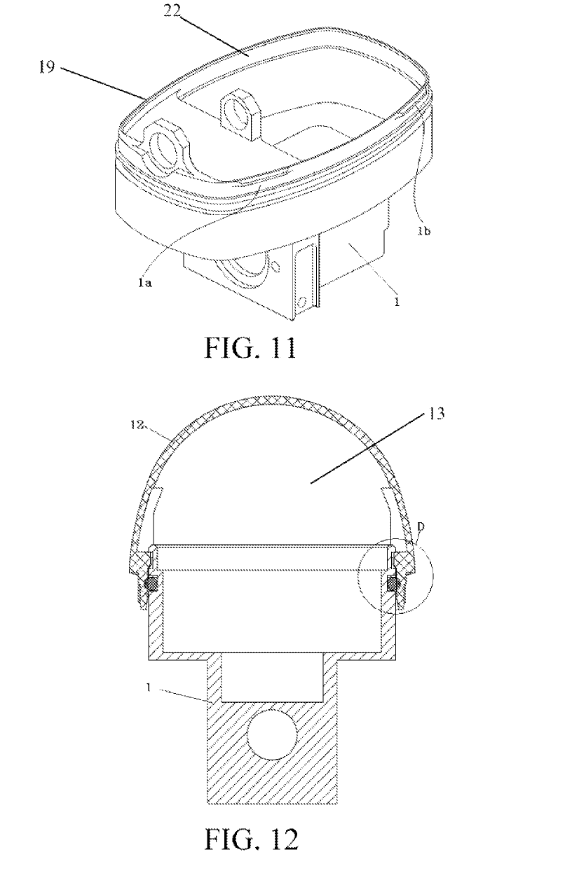

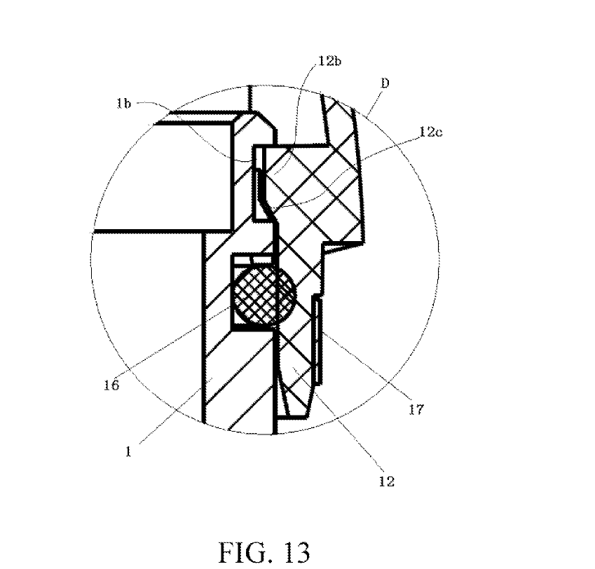

As shown in FIGS. 3 to 13, a 3D mechanical probe according to an embodiment includes an acoustic window 12, a base 1, an acoustic head 10, a driving power source, and a transmission mechanism. The acoustic window 12 and the base 1 are fixedly connected to form an enclosed space 13. The acoustic head 10 is disposed in the enclosed space 13. The enclosed space 13 is filled with a coupling liquid for conducting a sonic wave. The driving power source is used for providing power, and the driving power source drives the acoustic head 10 to swing inside the enclosed space 13 through the transmission mechanism.

The driving power source is, for example, a stepping motor 2, and the stepping motor 2 is fixed to the base 1 through a motor support 3. The transmission mechanism includes a driving synchronous belt wheel 4, a synchronous belt 6, a driven synchronous belt wheel 5, a driving shaft 7, a rope 8, a driven wheel 9, and an acoustic-head rotating shaft 11. The driving synchronous belt wheel 4 transfers an output motion of the stepping motor 2 to the driven synchronous belt wheel 5 through the synchronous belt 6. The driven synchronous belt wheel 5 is fixedly connected to the driving shaft 7. The driving shaft 7 is supported on the base 1 and is capable of free rotation. The driving shaft 7 drives the driven wheel 9 to swing through the rope 8. The driven wheel 9 is supported on the base 1 through the acoustic-head rotating shaft 11 and is capable of free rotation. The acoustic head 10 is fixedly connected to the driven wheel 9, and when the stepping motor 2 is in operation, the acoustic head 10 swings inside the enclosed space 13.

In one embodiment, the 3D mechanical probe may further include a seal ring 16. The acoustic window 12 has a first matching wall 18 at a bottom portion thereof. The base 1 has a second matching wall 19 at a top portion thereof. The first matching wall 18 is sleeved on the second matching wall 19. The first matching wall 18 has a first outer surface 20 and a first inner surface 21 surrounding the second matching wall 19, and the second matching wall 19 has a second outer surface 22 corresponding to the first inner surface 21. The second outer surface 22 of the base 1 is provided with a recessed annular seal groove 23. The seal ring 16 is annular and elastic. The seal ring 16 is disposed in the seal groove 23, and the seal ring 16 has a wall thickness larger than a depth of the seal groove 23. The wall thickness refers to a dimension of a section of the seal ring in the direction of an applied force. The acoustic window 12 tightly compresses the seal ring 16 into the seal groove 23 of the base 1 and causes the seal ring 16 to have a certain amount of compressive deformation, and an entire circumference of the seal ring 16 is deformed due to compression, thereby ensuring the sealing of the enclosed space 13 and achieving the effect of sealing the coupling liquid.

As the compressed seal ring 16 generates a counter force against the acoustic window 12, and the counter force may lead to expansive deformation of the acoustic window 12, the 3D mechanical probe may further have a restricting body, in order to restrict the deformation. The restricting body may be a readily deformable restricting collar 17 having a relatively fixed perimeter and tightly sleeved on the first outer surface 20 of the acoustic window 12. The restricting collar 17 can prevent the acoustic window 12 from being expanded, thereby ensuring a sufficient amount of compressive deformation of the seal ring 16 required for sealing.

In one embodiment, the restricting collar 17 may be made of a readily deformable metal sheet. Two ends of the metal sheet are respectively a head portion 17a and a tail portion 17b, and the head portion 17a and the tail portion 17b are connected and welded at the connection C to form a circular restricting collar. The head portion and the tail portion may be connected by butting or joining. In one embodiment, when connected by butting, the head portion 17a and the tail portion 17b have no overlapping portion, which can ensure a basically uniform wall thickness on the entire circumference of the restricting collar 17, so that the assembly space required is not too large, and the entire circumference of the restricting collar 17 can be in good contact with the acoustic window 12, thereby ensuring reliable sealing of the entire circumference.

When connected by joining, the head portion 17a and the tail portion 17b have an overlapping portion, and after welding, the strength of the overlapping portion is enhanced. The wall thickness of the metal sheet may be 0.1 mm, so that the metal sheet occupies a small space in the direction of the wall thickness and can provide a necessary structural space for other parts; the wall thickness of the metal sheet may also be designed as required. As the metal sheet has a low material cost and the cost of welding is also low, the cost of forming the restricting collar by connecting and welding the head and tail portions of the metal sheet is low. The restricting collar may also be a circular member formed by integral molding, e.g., cast molding. For the restricting collar, generally only the perimeter of the restricting collar needs to be controlled during manufacturing, and the shape does not need to be controlled, so that the manufacturing difficulty and cost can be reduced.

In order to ensure a reliable connection between the base 1 and the acoustic window 12, the first inner surface 21 of the first matching wall 18 of the acoustic window may be provided with two protruding barbs 12a and 12b serving as first buckling portions; each barb has a guiding surface 12c, and the guiding surface 12c may be a chamfer. The second outer surface 22 of the second matching wall 19 of the base is provided with two recessed buckling slots 1a and 1b serving as second buckling portions, and the buckling slots may be located above the seal groove. As the acoustic window easily deforms, the restricting collar 17 and the acoustic window 12 are assembled first, and then the acoustic window 12 is pressed downward. At this time, the guiding surfaces 12c of the barbs of the acoustic window 12 have a certain guiding effect, and gradually expand the acoustic window outward, and the barbs 12b are buckled in the buckling slots 1b upon entering the buckling slots 1b, so that the acoustic window 12 does not easily depart from the base 1, thereby ensuring the reliable connection between the acoustic window 12 and the base 1. The first matching wall may also be provided with one or more than two first buckling portions, and correspondingly, the second matching wall is provided with one or more than two second buckling portions.

The seal ring may be elastic and may be disposed between the first matching wall of the acoustic window and the second matching wall of the base, and the seal ring may have a certain amount of compressive deformation, so that the sealing of the enclosed space between the acoustic window and the base can be prevented, thereby preventing leakage of the coupling liquid. The first matching wall may be provided with a recessed seal groove, the seal ring is embedded in the seal groove, and the seal ring has a wall thickness larger than a depth of the seal groove. The second matching wall may be provided with a seal groove, or the first matching wall and the second matching wall are each provided with a seal groove, and the depth of the two seal grooves (that is, a distance between bottoms of the two seal grooves) is smaller than the wall thickness of the seal ring.

The acoustic window and the base may be fixedly connected, and the fixed connection manner may be a snap-fit engagement. For example, a first buckling portion is disposed on the first matching wall of the acoustic window and a second buckling portion is disposed on the second matching wall of the base; the first buckling portion and the second buckling portion are in a snap-fit engagement. The first buckling portion and the second buckling portion may also be disposed at other positions of the acoustic window and other positions of the base, respectively. The fixed connection manner between the acoustic window and the base may also be close fit; for example, the dimensions of the first matching wall and the second matching wall are configured so as to achieve a reliable connection through frictional cooperation between the two. The fixed connection manner between the acoustic window and the base may also be adhesive bonding; that is, an adhesive layer may be disposed between the first matching wall and the second matching wall, and in order to prevent the coupling liquid from contact with the adhesive layer, the adhesive layer may be located below the seal ring. The fixed connection manner between the acoustic window and the base may also be fastener connection; for example, a threaded fastener is used for connection. The fixed connection manner between the acoustic window and the base may also be other manners capable of reliably connecting the acoustic window and the base.

As a seal ring is disposed between the first matching wall of the acoustic window and the second matching wall of the base, and the seal ring may expand the acoustic window and cause deformation of the acoustic window, a restricting body may be disposed to restrict the deformation. The restricting body may be a circular member sleeved on the first matching wall, or a plurality of structures discretely distributed and pressed on the acoustic window to prevent deformation of the acoustic window.

The 3D mechanical probe may include a driving power source, a transmission mechanism, and an acoustic head, and the driving power source drives the acoustic head to swing inside the enclosed space between the acoustic window and the base through the transmission mechanism. The driving power source may be a motor or other mechanisms capable of providing power. The transmission mechanism may include a synchronous belt transmission mechanism, or may also include a gear transmission mechanism, a link mechanism, or other mechanisms capable of receiving the power and driving the acoustic head to swing.

The above descriptions are merely specific embodiments of the present disclosure, and are not intended to limit the scope of the invention. It is apparent to those of ordinary skill in the art that modifications and variations can be made without departing from the scope of the invention.

What is claimed is:

1. A three-dimensional (3D) mechanical probe comprising:
an acoustic window and a base, wherein the acoustic window and the base are fixedly connected to form an enclosed space, the acoustic window has a first matching wall, the base has a second matching wall, and the first matching wall is sleeved on the second matching wall; and
a restricting body and an elastic seal ring, wherein the seal ring is disposed between the first matching wall and the second matching wall and is tightly compressed and deformed, and the restricting body restricts deformation of the acoustic window.

2. The 3D mechanical probe according to claim 1, wherein the restricting body is a readily deformable restricting collar, and the restricting collar is tightly sleeved on the first matching wall.

3. The 3D mechanical probe according to claim 2, wherein two ends of the restricting collar are respectively a head portion and a tail portion, and the head portion and the tail portion are joined or butted and welded together.

4. The 3D mechanical probe according to claim 1, wherein at least one of the first matching wall and the second matching wall is provided with a recessed seal groove, the seal ring is disposed in the seal groove, and the seal ring has a wall thickness larger than a depth of the seal groove.

5. The 3D mechanical probe according to claim 1, wherein the acoustic window is in a snap-fit engagement with the base.

6. The 3D mechanical probe according to claim 5, wherein the first matching wall is provided with a first buckling portion, the second matching wall is provided with a second buckling portion, and the first buckling portion is in snap-fit engagement with the second buckling portion.

7. The 3D mechanical probe according to claim 6, wherein the first matching wall has an inner surface surrounding the base, the second matching wall has an outer surface corresponding to the inner surface, the seal ring is located between the inner surface and the outer surface, the first buckling portion protrudes from the inner surface, and the second buckling portion is recessed in the outer surface.

8. The 3D mechanical probe according to claim 7, wherein at least one of the first buckling portion and the second buckling portion has a guiding surface.
